(19) United States
(12) Patent Application Publication
(10) Pub. No.: US 2024/0269153 A1
(43) Pub. Date: Aug. 15, 2024

(54) BIOACTIVE BIPHASIC COMPOSITIONS

(71) Applicant: NATURAL EXTRACTION SYSTEMS, LLC, Boulder, CO (US)

(72) Inventor: C. Russell THOMAS, Boulder, CO (US)

(21) Appl. No.: 18/565,033

(22) PCT Filed: May 27, 2022
(86) PCT No.: PCT/US2022/031456

§ 371 Date: Nov. 28, 2023

Related U.S. Application Data
(60) Provisional application No. 63/194,816, filed on May 28, 2021.
(60) Provisional application No. 63/254,438, filed on Oct. 11, 2021.

(51) Int. Cl. A61K 31/00; A61K 9/00; A61K 9/14; A61K 36/9066

(52) U.S. Cl. CPC A61K 31/658; A61K 9/0053; A61K 9/14; A61K 36/9066

(57) ABSTRACT

Various aspects of this disclosure relate to excipients comprising a solid phase and a liquid phase, wherein the solid phase has a surface-area-to-volume ratio of at least 500 per meter, which improves the bioavailability and pharmacokinetics of various hydrophobic ingredients. Compositions comprising such excipients can be utilized to disperse a hydrophobic ingredient in a beverage, which improves the bioavailability and pharmacokinetics of the dispersed hydrophobic ingredient.

### CROSS-REFERENCE TO RELATED APPLICATIONS

This patent application claims priority to United States Provisional Patent Application No. 63/194,816, filed May 28, 2021, and U.S. Provisional Patent Application No. 63/254,438, filed Oct. 11, 2021, each of which is incorporated by reference in its entirety.

### BACKGROUND

The “Rule of Five” states that a druglike agent generally has a common logarithm of its octanol-water partition coefficient that is no greater than 5. A generally-applicable strategy to overcome this limitation of the Rule of Five is desirable.

### SUMMARY

Bioactive agents that possess octanol-water partition coefficients significantly greater than 1 generally display poor oral bioavailability and pharmacokinetics. Such agents favor aggregating or partitioning into a lipid phase in the gastrointestinal tract rather than assimilation into the body. The small intestine contains lipase enzymes that metabolize lipids to break apart lipid phases and release bioactive agents for absorption, but significant portions of hydrophobic agents are excreted. Upon entering the hepatic-portal circulation, bioactive agents that the body absorbs are directed into the liver where cytochrome P450 enzymes and other enzymes generally oxidize hydrophobic agents.

The descriptions that follow describe methods to disperse hydrophobic agents in biphasic compositions using excipients that readily dissolve in water. The oral administration of such biphasic compositions results in the dissolution of the excipients. When the hydrophobic agents are sufficiently separated in the biphasic composition, then the dissolution of the excipients results in the partitioning of the hydrophobic agents into the gastrointestinal epithelium rather than aggregation or partitioning into a lipid phase, which generally improves oral bioavailability and pharmacokinetics. When a biphasic composition is formulated such that dissolution of the excipients occurs in the buccal cavity, for example, then oral administration can bypass the hepatic-portal circulation to further improve oral bioavailability and pharmacokinetics.

The biphasic compositions of the disclosure are amenable to the formulation of beverage powders in which a “powder” is a biphasic composition. Such beverage powders can be combined with water or other beverage to disperse a hydrophobic ingredient in the water or other beverage.

### SUMMARY

Various aspects of the disclosure relate to a composition, comprising a solid phase and a liquid phase, wherein the solid phase has a surface-area-to-volume ratio that is greater than 500 per meter; the liquid phase consists of ingredients; each ingredient of the ingredients of the liquid phase has a concentration by mass in the liquid phase; the ingredients of the liquid phase comprise a solvent; the concentration by mass of the solvent in the liquid phase is greater than the concentration by mass of any other ingredient of the liquid phase; and the composition comprises the solid phase at a greater concentration by mass than the liquid phase.

“Comprise” refers to an open set, for example, such that a composition comprising a solid phase and a liquid phase can also comprise a gas phase.

“Consists” refers to a closed set, for example, such that a liquid phase that consists of ingredients cannot also comprise a chemical species other than the ingredients.

In some embodiments, the composition comprises the liquid phase at a concentration of at least 0.1 percent and no greater than 10 percent by mass; and the composition comprises the solid phase at a concentration of at least 90 percent and no greater than 99.9 percent by mass.

In some embodiments, a composition comprises an active agent.

Various aspects of this disclosure relate to a composition described anywhere in this disclosure, for use as a medicament.

Various aspects of this disclosure relate to a composition described anywhere in this disclosure, for use to manufacture a medicament.

Various aspects of this disclosure relate to a method to administer an active ingredient, comprising providing a composition as described anywhere in this disclosure, wherein the composition comprises the active ingredient; and orally administering the composition.

In some embodiments, the method comprises combining the composition with a second composition prior to administering the composition, wherein the second composition is a liquid that comprises water at a concentration of at least 50 molar.

Various aspects of this disclosure relate to a method to prepare a beverage, comprising providing a composition as described anywhere in this disclosure and combining the composition with a second composition, wherein the second composition is a liquid that comprises water at a concentration of at least 50 molar.

In some embodiments, the composition comprises an active agent; the active agent is dissolved in the liquid phase of the composition; and combining the composition with a second composition results in phase separation of the active agent from the water.

In some embodiments, the method comprises combining at least 100 milligrams and no greater than 5 grams of the composition with at least 10 grams and no greater than 800 grams of the second composition.

In some embodiments, the second composition is a beverage.

In some embodiments, a human performs the method; and the human consumes the combination of the composition and the second composition by drinking it.

Various aspects of this disclosure relate to a container that contains at least 100 milligrams and no greater than 5 grams of a composition as described anywhere in this disclosure.

In some embodiments, the container is a sachet; and the composition is hermetically sealed within the container.

### DETAILED DESCRIPTION

Various aspects of the disclosure relate to a composition, comprising a solid phase and a liquid phase, wherein the solid phase has a surface-area-to-volume ratio that is greater than 500 per meter; the liquid phase consists of ingredients; each ingredient of the ingredients of the liquid phase has a concentration by mass in the liquid phase; the ingredients of the liquid phase comprise a solvent; the concentration by mass of the solvent in the liquid phase is greater than the concentration by mass of any other ingredient of the liquid phase; and the composition comprises the solid phase at a greater concentration by mass than the liquid phase.

In some embodiments, the solid phase consists of ingredients; and at least 90 percent by mass of the ingredients of the solid phase have solubilities in water at 21 degrees Celsius of at least 100 grams per liter.

In some embodiments the solid phase consists of ingredients; and the ingredients of the solid phase comprise carbohydrates.

In some embodiments the carbohydrates consist of one or more polysaccharides, sulfated polysaccharides, oligosaccharides, disaccharides, monosaccharides, and sugar alcohols.

In some embodiments, the carbohydrates consist of one or more of alginic acid, alginate, propylene glycol alginate, inulin, arabinogalactan, lignosulfonate, carrageenan, furcelleran, pullulan, glucomannan, gellan gum, gum arabic, gum ghatti, guar gum, gum karaya, tara gum, tragacanth, xanthan gum, carboxymethylcellulose, hydroxyethyl cellulose, ethylhydroxyethylcellulose, hydroxypropyl cellulose, hydroxypropyl methylcellulose, modified starch, dextrin, polydextrose, pectin, beta-glucan, lactobionic acid, lactobionate, isomalt, erythritol, threitol, arabitol, xylitol, ribitol, mannitol, sorbitol, galactitol, fucitol, iditol, inositol, volemitol, sucrose, lactose, maltose, trehalose, maltitol, glucose, fructose, galactose, arabinose, ribose, xylose, allulose, sorbose, tagatose, fucose, mannose, and hydrogenated starch hydrolysate.

In some embodiments, the carbohydrates consist of sugar alcohols.

In some embodiments, the carbohydrates comprise butane-1,2,3,4-tetrol.

In some embodiments, the liquid phase comprises butane-1,2,3,4-tetrol and one or both of 1,3,4-trihydroxy butane-2-oxide and 2,3,4-trihydroxy butane-1-oxide at a molar ratio of at least 100:1 and no greater than 10,000,000:1 (butane-1,2,3,4-tetrol:1,3,4-trihydroxy butane-2-oxide and 2,3,4-trihydroxy butane-1-oxide).

In some embodiments, the butane-1,2,3,4-tetrol comprises one or both of erythritol and threitol.

In some embodiments, the carbohydrates comprise pentane-1,2,3,4,5-pentol.

In some embodiments, the liquid phase comprises pentane-1,2,3,4,5-pentol and one, two, or each of 1,2,4,5-tetrahydroxypentane-3-oxide, 1,3,4,5-tetrahydroxypentane-2-oxide, and 2,3,4,5-tetrahydroxypentane-1-oxide at a molar ratio of at least 100:1 and no greater than 10,000,000:1 (pentane-1,2,3,4,5-pentol:1,2,4,5-tetrahydroxypentane-3-oxide, 1,3,4,5-tetrahydroxy pentane-2-oxide, and 2,3,4,5-tetrahydroxypentane-1-oxide).

In some embodiments, the pentane-1,2,3,4,5-pentol comprises one, two, or each of arabitol, xylitol, and ribitol.

In some embodiments, the pentane-1,2,3,4,5-pentol is xylitol.

In some embodiments, the carbohydrates comprise hexane-1,2,3,4,5,6-hexol.

In some embodiments, the liquid phase comprises hexane-1,2,3,4,5,6-hexol and one, two, or each of 1,2,4,5,6-pentahydroxyhexane-3-oxide, 1,3,4,5,6-pentahydroxyhexane-2-oxide, and 2,3,4,5,6-pentahydroxyhexane-1-oxide at a molar ratio of at least 100:1 and no greater than 10,000,000:1 (hexane-1,2,3,4,5,6-hexol:1,2,4,5,6-pentahydroxy hexane-3-oxide, 1,3,4,5,6-pentahydroxyhexane-2-oxide, and 2,3,4,5,6-pentahydroxy hexane-1-oxide).

In some embodiments, the hexane-1,2,3,4,5,6-hexol comprises one, two, three, four, or each of mannitol, sorbitol, galactitol, fucitol, and iditol.

In some embodiments, the carbohydrates comprise sucrose.

In some embodiments, the carbohydrates comprise maltose.

In some embodiments, the carbohydrates comprise trehalose.

In some embodiments, the carbohydrates comprise maltitol.

In some embodiments, the carbohydrates comprise glucose.

In some embodiments, the carbohydrates comprise fructose.

In some embodiments, the carbohydrates comprise allulose.

In some embodiments, at least 90 percent of the ingredients of the solid phase consist of the carbohydrates by mass.

In some embodiments, the composition comprises the liquid phase at a concentration of at least 0.1 percent and no greater than 10 percent by mass; and the composition comprises the solid phase at a concentration of at least 90 percent and no greater than 99.9 percent by mass.

In some embodiments, the composition comprises the liquid phase at a concentration of at least 0.5 percent and no greater than 15 percent by mass; and the composition comprises the solid phase at a concentration of at least 85 percent and no greater than 99.5 percent by mass.

In some embodiments, the composition comprises at least 0.6 and no greater than 6 calories of food energy per gram of the composition.

In some embodiments, the solvent is miscible with water.

In some embodiments, the solvent is acetic acid; acetone; 1-butanol; 2-butanol; butan-2-one; 1,3-butanediol; dimethyl sulfoxide; ethanol; ethyl acetate; ethyl formate; formic acid; isoamyl alcohol; isobutanol; isopropyl acetate; methyl acetate; 1-pentanol; 1-propanol; 2-propanol; propane-1,2-diol; propane-1,3-diol; propane-1,2,3-triol; propylacetate; or triethylamine.

In some embodiments, the solvent is ethanol.

In some embodiments, the solvent is propane-1,2-diol.

In some embodiments, the solvent is propane-1,2,3-triol.

In some embodiments, the solvent is water.

In some embodiments, the ingredients of the liquid phase comprise a cosolvent; and the solvent and the cosolvent are different ingredients.

In some embodiments, the cosolvent is acetic acid; acetone; 1-butanol; 2-butanol; butan-2-one; 1,3-butanediol; dimethyl sulfoxide; ethanol; ethyl acetate; ethyl formate; formic acid; isoamyl alcohol; isobutanol; isopropyl acetate; methyl acetate; 1-pentanol; 1-propanol; 2-propanol; propane-1,2-diol; propane-1,3-diol; propane-1,2,3-triol; propylacetate; or triethylamine.

In some embodiments, the cosolvent is ethanol.

In some embodiments, the solvent is propane-1,2-diol and the cosolvent is ethanol.

In some embodiments, the liquid phase comprises propane-1,2-diol and one or both of 1-hydroxypropane-2-oxide and 2-hydroxypropane-1-oxide at a molar ratio of at least 100:1 and no greater than 10,000,000:1 (propane-1,2-diol:1-hydroxypropane-2-oxide and 2-hydroxypropane-1-oxide);

In some embodiments, the liquid phase comprises ethanol and ethoxide at a molar ratio of at least 100:1 and no greater than 10,000,000:1.

In some embodiments, the liquid phase comprises water and hydroxide; wherein the water and the hydroxide are solutes that are dissolved in the solvent of the liquid phase at a molar ratio of at least 100:1 and no greater than 10,000,000:1.

In some embodiments, the liquid phase comprises a cation; wherein the cation is a solute that is dissolved in the solvent of the liquid phase.

In some embodiments, the cation is ammonium; protonated ethanolamine; choline; protonated lysine; protonated arginine; protonated sphingosine; lithium cation (“Li+”); sodium cation (“Na+”); potassium cation (“K+”); magnesium cation (“Mg++”); calcium cation (“Ca++”); zinc cation (“Zn++”); manganese cation (“Mn++”); iron (II) cation (“Fe++”); iron (III) cation (“Fe+++”); copper (I) cation (“Cu+”); or copper (II) cation (“Cu++”).

In some embodiments, the cation is potassium cation.

In some embodiments, the cation is sodium cation.

In some embodiments, the cation is dissolved in the solvent of the liquid phase at a concentration of at least 100 micromolar and no greater than 500 millimolar.

In some embodiments, the ingredients of the liquid phase comprise an active ingredient; and the active ingredient is a solute that is dissolved in the solvent of the liquid phase.

In some embodiments, the active ingredient is dissolved in the solvent of the liquid phase at a concentration of at least 1 millimolar and no greater than 500 millimolar.

In some embodiments, the active ingredient is dissolved in the solvent of the liquid phase at a concentration of at least 200 micromolar and no greater than 250 millimolar.

In some embodiments, the active ingredient has a solubility in water at 37 degrees Celsius; and the active ingredient is dissolved in the solvent of the liquid phase at a concentration that is at least 100 percent greater than the solubility of the active ingredient in water at 37 degrees Celsius.

In some embodiments, the active ingredient has an octanol-water partition coefficient of at least 1.

In some embodiments, the active ingredient has an octanol-water partition coefficient of at least 100.

In some embodiments, the active ingredient has an octanol-water partition coefficient of at least 10,000.

In some embodiments, the active ingredient has an octanol-water partition coefficient of at least 100 and no greater than 1,000,000.

In some embodiments, the active ingredient is 2-geranyl-3-hydroxy-5-pentylphenolate.

In some embodiments, the active ingredient is 3-hydroxy-2-(6-isopropenyl-3-methylcyclohex-2-enyl)-5-pentylphenolate.

In some embodiments, the active ingredient is 3-hydroxy-2-(6-isopropenyl-3-methylcyclohex-3-enyl)-5-pentylphenolate.

In some embodiments, the active ingredient is 6,6,9-trimethyl-3-pentyl-6a, 7,8,10a-tetrahydro-6H-benzo[c]chromene-1-oxide.

In some embodiments, the active ingredient is 6,6,9-trimethyl-3-pentyl-6a, 7,10,10a-tetrahydro-6H-benzo[c]chromene-1-oxide.

In some embodiments, the active ingredient is 6,6,9-trimethyl-3-pentyl-6H-benzo[c]chromene-1-oxide.

In some embodiments, the active ingredient is 2-geranyl-5-pentylbenzene-1,3-diol.

In some embodiments, the active ingredient is 2-(6-isopropenyl-3-methylcyclohex-2-enyl)-5-pentylbenzene-1,3-diol.

In some embodiments, the active ingredient is 2-(6-isopropenyl-3-methylcyclohex-3-enyl)-5-pentylbenzene-1,3-diol.

In some embodiments, the active ingredient is 6,6,9-trimethyl-3-pentyl-6a, 7,8,10a-tetrahydro-6H-benzo[c]chromene-1-ol.

In some embodiments, the active ingredient is 6,6,9-trimethyl-3-pentyl-6a, 7,10,10a-tetrahydro-6H-benzo[c]chromene-1-ol.

In some embodiments, the active ingredient is 6,6,9-trimethyl-3-pentyl-6H-benzo[c]chromene-1-ol.

In some embodiments, the active ingredient is 2-geranyl-3-hydroxy-5-propylphenolate.

In some embodiments, the active ingredient is 3-hydroxy-2-(6-isopropenyl-3-methylcyclohex-2-enyl)-5-propylphenolate.

In some embodiments, the active ingredient is 3-hydroxy-2-(6-isopropenyl-3-methylcyclohex-3-enyl)-5-propylphenolate.

In some embodiments, the active ingredient is 6,6,9-trimethyl-3-propyl-6a, 7,8,10a-tetrahydro-6H-benzo[c]chromene-1-oxide.

In some embodiments, the active ingredient is 6,6,9-trimethyl-3-propyl-6a, 7,10,10a-tetrahydro-6H-benzo[c]chromene-1-oxide.

In some embodiments, the active ingredient is 6,6,9-trimethyl-3-propyl-6H-benzo[c]chromene-1-oxide.

In some embodiments, the active ingredient is 2-geranyl-5-propylbenzene-1,3-diol.

In some embodiments, the active ingredient is 2-(6-isopropenyl-3-methylcyclohex-2-enyl)-5-propylbenzene-1,3-diol.

In some embodiments, the active ingredient is 2-(6-isopropenyl-3-methylcyclohex-3-enyl)-5-propylbenzene-1,3-diol.

In some embodiments, the active ingredient is 6,6,9-trimethyl-3-propyl-6a, 7,8,10a-tetrahydro-6H-benzo[c]chromene-1-ol.

In some embodiments, the active ingredient is 6,6,9-trimethyl-3-propyl-6a, 7,10,10a-tetrahydro-6H-benzo[c]chromene-1-ol.

In some embodiments, the active ingredient is 6,6,9-trimethyl-3-propyl-6H-benzo[c]chromene-1-ol.

In some embodiments, the active ingredient is curcumin.

In some embodiments, the active ingredient is a flavonoid.

In some embodiments, the active ingredient is an anthocyanin or anthocyanidin.

In some embodiments, the active ingredient is epigallocatechin gallate.

In some embodiments, the active ingredient is a tannin.

In some embodiments, the active ingredient is a cannabinoid.

In some embodiments, the active ingredient is tetrahydrocannabinol.

In some embodiments, the active ingredient is tetrahydrocannabivarin.

In some embodiments, the active ingredient is delta8-tetrahydrocannabinol.

In some embodiments, the active ingredient is delta8-tetrahydrocannabivarin.

In some embodiments, the active ingredient is cannabidiol.

In some embodiments, the active ingredient is cannabidivarin.

In some embodiments, the active ingredient is cannabigerol.

In some embodiments, the active ingredient is cannabigerovarin.

In some embodiments, the active ingredient is cannabinol.

In some embodiments, the active ingredient is cannabivarin.

In some embodiments, the composition lacks triglycerides at a concentration greater than 0.1 percent by mass.

In some embodiments, the solid phase comprises carbonate; and the liquid phase comprises carbonate and bicarbonate.

In some embodiments, the composition is formulated for oral administration.

Various aspects of this disclosure relate to a composition described anywhere in this disclosure, for use to manufacture a medicament.

Various aspects of this disclosure relate to a method to administer an active ingredient, comprising providing a composition as described anywhere in this disclosure, wherein the composition comprises the active ingredient; and orally administering the composition.

In some embodiments, the method comprises combining the composition with a second composition prior to administering the composition, wherein the second composition is a liquid that comprises water at a concentration of at least 50 molar.

Various aspects of this disclosure relate to a method to prepare a beverage, comprising providing a composition as described anywhere in this disclosure and combining the composition with a second composition, wherein the second composition is a liquid that comprises water at a concentration of at least 50 molar.

In some embodiments, the composition comprises an active agent; the active agent is dissolved in the liquid phase of the composition; and combining the composition with a second composition results in phase separation of the active agent from the water.

In some embodiments, the method comprises combining at least 100 milligrams and no greater than 5 grams of the composition with at least 10 grams and no greater than 800 grams of the second composition.

In some embodiments, the second composition is a beverage.

In some embodiments, a human performs the method; and the human consumes the combination of the composition and the second composition by drinking it.

Various aspects of this disclosure relate to a container that contains at least 100 milligrams and no greater than 5 grams of a composition as described anywhere in this disclosure.

In some embodiments, the container is a sachet; and the composition is hermetically sealed within the container.

In some embodiments, the container comprises metallized polyethylene terephthalate that is metallized with aluminum.

What is claimed is:

1. A composition, comprising a solid phase and a liquid phase, wherein the solid phase has a surface-area-to-volume ratio that is greater than 500 per meter; the liquid phase consists of ingredients; each ingredient of the ingredients of the liquid phase has a concentration by mass in the liquid phase; the ingredients of the liquid phase comprise a solvent; the concentration by mass of the solvent in the liquid phase is greater than the concentration by mass of any other ingredient of the liquid phase; and the composition comprises the solid phase at a greater concentration by mass than the liquid phase.

2. The composition of claim 1, wherein the solid phase consists of ingredients; and at least 90 percent by mass of the ingredients of the solid phase have solubilities in water at 21 degrees Celsius of at least 100 grams per liter.

3. The composition of claim 1 or 2, wherein the solid phase consists of ingredients; and the ingredients of the solid phase comprise carbohydrates.

4. The composition of claim 3, wherein the carbohydrates consist of one or more polysaccharides, sulfated polysaccharides, oligosaccharides, disaccharides, monosaccharides, and sugar alcohols.

5. The composition of claim 3 or 4, wherein the carbohydrates consist of one or more of alginic acid, alginate, propylene glycol alginate, inulin, arabinogalactan, lignosulfonate, carrageenan, furcelleran, pullulan, glucomannan, gellan gum, gum arabic, gum ghatti, guar gum, gum karaya, tara gum, tragacanth, xanthan gum, carboxymethylcellulose, hydroxyethyl cellulose, ethylhydroxyethylcellulose, hydroxypropyl cellulose, hydroxypropyl methylcellulose, modified starch, dextrin, polydextrose, pectin, beta-glucan, lactobionic acid, lactobionate, isomalt, erythritol, threitol, arabitol, xylitol, ribitol, mannitol, sorbitol, galactitol, fucitol, iditol, inositol, volemitol, sucrose, lactose, maltose, trehalose, maltitol, glucose, fructose, galactose, arabinose, ribose, xylose, allulose, sorbose, tagatose, fucose, mannose, and hydrogenated starch hydrolysate.

6. The composition of any one of claims 3-5, wherein the carbohydrates consist of sugar alcohols.

7. The composition of any one of claims 3-6, wherein the carbohydrates comprise butane-1,2,3,4-tetrol.

8. The composition of any one of claims 3-7, wherein the liquid phase comprises butane-1,2,3,4-tetrol and one or both of 1,3,4-trihydroxy butane-2-oxide and 2,3,4-trihydroxy butane-1-oxide at a molar ratio of at least 100:1 and no greater than 10,000,000:1 (butane-1,2,3,4-tetrol:1,3,4-trihydroxy butane-2-oxide and 2,3,4-trihydroxy butane-1-oxide).

9. The composition of claim 7 or 8, wherein the butane-1,2,3,4-tetrol comprises one or both of erythritol and threitol.

10. The composition of any one of claims 3-9, wherein the carbohydrates comprise pentane-1,2,3,4,5-pentol.

11. The composition of any one of claims 3-10, wherein the liquid phase comprises pentane-1,2,3,4,5-pentol and one, two, or each of 1,2,4,5-tetrahydroxypentane-3-oxide, 1,3,4,5-tetrahydroxypentane-2-oxide, and 2,3,4,5-tetrahydroxypentane-1-oxide at a molar ratio of at least 100:1 and no greater than 10,000,000:1 (pentane-1,2,3,4,5-pentol:1,2,4,5-tetrahydroxypentane-3-oxide, 1,3,4,5-tetrahydroxypentane-2-oxide, and 2,3,4,5-tetrahydroxypentane-1-oxide).

12. The composition of claim 10 or 11, wherein the pentane-1,2,3,4,5-pentol comprises one, two, or each of arabitol, xylitol, and ribitol.

13. The composition of any one of claims 10-12, wherein the pentane-1,2,3,4,5-pentol is xylitol.

14. The composition of any one of claims 3-13, wherein the carbohydrates comprise hexane-1,2,3,4,5,6-hexol.

15. The composition of any one of claims 3-14, wherein the liquid phase comprises hexane-1,2,3,4,5,6-hexol and one, two, or each of 1,2,4,5,6-pentahydroxyhexane-3-oxide, 1,3,4,5,6-pentahydroxyhexane-2-oxide, and 2,3,4,5,6-pentahydroxyhexane-1-oxide at a molar ratio of at least 100:1 and no greater than 10,000,000:1 (hexane-1,2,3,4,5,6-hexol:1,2,4,5,6-pentahydroxyhexane-3-oxide, 1,3,4,5,6-pentahydroxyhexane-2-oxide, and 2,3,4,5,6-pentahydroxyhexane-1-oxide).

16. The composition of claim 14 or 15, wherein the hexane-1,2,3,4,5,6-hexol comprises one, two, three, four, or each of mannitol, sorbitol, galactitol, fucitol, and iditol.

17. The composition of any one of claims 3-16, wherein the carbohydrates comprise sucrose.

18. The composition of any one of claims 3-17, wherein the carbohydrates comprise maltose.

19. The composition of any one of claims 3-18, wherein the carbohydrates comprise trehalose.

20. The composition of any one of claims 3-19, wherein the carbohydrates comprise maltitol.

21. The composition of any one of claims 3-20, wherein the carbohydrates comprise glucose.

22. The composition of any one of claims 3-21, wherein the carbohydrates comprise fructose.

23. The composition of any one of claims 3-22, wherein the carbohydrates comprise allulose.

24. The composition of any one of claims 3-23, wherein at least 90 percent of the ingredients of the solid phase consist of the carbohydrates by mass.

25. The composition of any one of claims 1-24, wherein the composition comprises the liquid phase at a concentration of at least 0.1 percent and no greater than 10 percent by mass; and the composition comprises the solid phase at a concentration of at least 90 percent and no greater than 99.9 percent by mass.

26. The composition of any one of claims 1-25, wherein the composition comprises the liquid phase at a concentration of at least 0.5 percent and no greater than 15 percent by mass; and the composition comprises the solid phase at a concentration of at least 85 percent and no greater than 99.5 percent by mass.

27. The composition of any one of claims 1-26, wherein the composition comprises at least 0.6 and no greater than 6 calories of food energy per gram of the composition.

28. The composition of any one of claims 1-27, wherein the solvent is miscible with water.

29. The composition of any one of claims 1-28, wherein the solvent is acetic acid; acetone; 1-butanol; 2-butanol; butan-2-one; 1,3-butanediol; dimethyl sulfoxide; ethanol; ethyl acetate; ethyl formate; formic acid; isoamyl alcohol; isobutanol; isopropyl acetate; methyl acetate; 1-pentanol; 1-propanol; 2-propanol; propane-1,2-diol; propane-1,3-diol; propane-1,2,3-triol; propylacetate; or triethylamine.

30. The composition of any one of claims 1-29, wherein the solvent is ethanol.

31. The composition of any one of claims 1-30, wherein the solvent is propane-1,2-diol.

32. The composition of any one of claims 1-31, wherein the solvent is propane-1,2,3-triol.

33. The composition of any one of claims 1-28, wherein the solvent is water.

34. The composition of any one of claims 1-33, wherein the ingredients of the liquid phase comprise a cosolvent; and the solvent and the cosolvent are different ingredients.

35. The composition of claim 34, wherein the cosolvent is acetic acid; acetone; 1-butanol; 2-butanol; butan-2-one; 1,3-butanediol; dimethyl sulfoxide; ethanol; ethyl acetate; ethyl formate; formic acid; isoamyl alcohol; isobutanol; isopropyl acetate; methyl acetate; 1-pentanol; 1-propanol; 2-propanol; propane-1,2-diol; propane-1,3-diol; propane-1,2,3-triol; propylacetate; or triethylamine.

36. The composition of claim 34 or 35, wherein the cosolvent is ethanol.

37. The composition of any one of claims 34-36, wherein the solvent is propane-1,2-diol and the cosolvent is ethanol.

38. The composition of any one of claims 1-37, wherein the liquid phase comprises propane-1,2-diol and one or both of 1-hydroxypropane-2-oxide and 2-hydroxypropane-1-oxide at a molar ratio of at least 100:1 and no greater than 10,000,000:1 (propane-1,2-diol:1-hydroxy propane-2-oxide and 2-hydroxypropane-1-oxide);

39. The composition of any one of claims 1-38, wherein the liquid phase comprises ethanol and ethoxide at a molar ratio of at least 100:1 and no greater than 10,000,000:1.

40. The composition of any one of claims 1-39, comprising water and hydroxide; wherein the water and the hydroxide are solutes that are dissolved in the solvent of the liquid phase at a molar ratio of at least 100:1 and no greater than 10,000,000:1.

41. The composition of any one of claims 1-40 comprising a cation; wherein the cation is a solute that is dissolved in the solvent of the liquid phase.

42. The composition of claim 41, wherein the cation is ammonium; protonated ethanolamine; choline; protonated lysine; protonated arginine; protonated sphingosine; lithium cation (“Li+”); sodium cation (“Na+”); potassium cation (“K+”); magnesium cation (“Mg++”); calcium cation (“Ca++”); zinc cation (“Zn++”); manganese cation (“Mn++”); iron (II) cation (“Fe++”); iron (III) cation (“Fe+++”); copper (I) cation (“Cu+”); or copper (II) cation (“Cu++”).

43. The composition of claim 41 or 42, wherein the cation is potassium cation.

44. The composition of any one of claims 41-43, wherein the cation is dissolved in the solvent of the liquid phase at a concentration of at least 100 micromolar and no greater than 500 millimolar.

45. The composition of any one of claims 1-44, wherein the ingredients of the liquid phase comprise an active ingredient; and the active ingredient is a solute that is dissolved in the solvent of the liquid phase.

46. The composition of claim 45, wherein the active ingredient is dissolved in the solvent of the liquid phase at a concentration of at least 1 millimolar and no greater than 500 millimolar.

47. The composition of claim 45 or 46, wherein the active ingredient is dissolved in the solvent of the liquid phase at a concentration of at least 200 micromolar and no greater than 250 millimolar.

48. The composition of any one of claims 45-47, wherein the active ingredient has a solubility in water at 37 degrees Celsius; and the active ingredient is dissolved in the solvent of the liquid phase at a concentration that is at least 100 percent greater than the solubility of the active ingredient in water at 37 degrees Celsius.

49. The composition of any one of claims 45-48, wherein the active ingredient has an octanol-water partition coefficient of at least 1.

50. The composition of any one of claims 45-49, wherein the active ingredient has an octanol-water partition coefficient of at least 100.

51. The composition of any one of claims 45-50, wherein the active ingredient has an octanol-water partition coefficient of at least 10,000.

52. The composition of any one of claims 45-51, wherein the active ingredient has an octanol-water partition coefficient of at least 100 and no greater than 1,000,000.

53. The composition of any one of claims 45-52, wherein the active ingredient is 2-geranyl-3-hydroxy-5-pentylphenolate.

54. The composition of any one of claims 45-52, wherein the active ingredient is 3-hydroxy-2-(6-isopropenyl-3-methylcyclohex-2-enyl)-5-pentylphenolate.

55. The composition of any one of claims 45-52, wherein the active ingredient is 3-hydroxy-2-(6-isopropenyl-3-methylcyclohex-3-enyl)-5-pentylphenolate.

56. The composition of any one of claims 45-52, wherein the active ingredient is 6,6,9-trimethyl-3-pentyl-6a, 7,8,10a-tetrahydro-6H-benzo[c]chromene-1-oxide.

57. The composition of any one of claims 45-52, wherein the active ingredient is 6,6,9-trimethyl-3-pentyl-6a,7,10,10a-tetrahydro-6H-benzo[c]chromene-1-oxide.

58. The composition of any one of claims 45-52, wherein the active ingredient is 6,6,9-trimethyl-3-pentyl-6H-benzo[c]chromene-1-oxide.

59. The composition of any one of claims 45-52, wherein the active ingredient is 2-geranyl-5-pentylbenzene-1,3-diol.

60. The composition of any one of claims 45-52, wherein the active ingredient is 2-(6-isopropenyl-3-methylcyclohex-2-enyl)-5-pentylbenzene-1,3-diol.

61. The composition of any one of claims 45-52, wherein the active ingredient is 2-(6-isopropenyl-3-methylcyclohex-3-enyl)-5-pentylbenzene-1,3-diol.

62. The composition of any one of claims 45-52, wherein the active ingredient is 6,6,9-trimethyl-3-pentyl-6a, 7,8,10a-tetrahydro-6H-benzo[c]chromene-1-ol.

63. The composition of any one of claims 45-52, wherein the active ingredient is 6,6,9-trimethyl-3-pentyl-6a,7,10,10a-tetrahydro-6H-benzo[c]chromene-1-ol.

64. The composition of any one of claims 45-52, wherein the active ingredient is 6,6,9-trimethyl-3-pentyl-6H-benzo[c]chromene-1-ol.

65. The composition of any one of claims 45-52, wherein the active ingredient is 2-geranyl-3-hydroxy-5-propylphenolate.

66. The composition of any one of claims 45-52, wherein the active ingredient is 3-hydroxy-2-(6-isopropenyl-3-methylcyclohex-2-enyl)-5-propylphenolate.

67. The composition of any one of claims 45-52, wherein the active ingredient is 3-hydroxy-2-(6-isopropenyl-3-methylcyclohex-3-enyl)-5-propylphenolate.

68. The composition of any one of claims 45-52, wherein the active ingredient is 6,6,9-trimethyl-3-propyl-6a,7,8,10a-tetrahydro-6H-benzo[c]chromene-1-oxide.

69. The composition of any one of claims 45-52, wherein the active ingredient is 6,6,9-trimethyl-3-propyl-6a, 7,10,10a-tetrahydro-6H-benzo[c]chromene-1-oxide.

70. The composition of any one of claims 45-52, wherein the active ingredient is 6,6,9-trimethyl-3-propyl-6H-benzo[c]chromene-1-oxide.

71. The composition of any one of claims 45-52, wherein the active ingredient is 2-geranyl-5-propylbenzene-1,3-diol.

72. The composition of any one of claims 45-52, wherein the active ingredient is 2-(6-isopropenyl-3-methylcyclohex-2-enyl)-5-propylbenzene-1,3-diol.

73. The composition of any one of claims 45-52, wherein the active ingredient is 2-(6-isopropenyl-3-methylcyclohex-3-enyl)-5-propylbenzene-1,3-diol.

74. The composition of any one of claims 45-52, wherein the active ingredient is 6,6,9-trimethyl-3-propyl-6a, 7,8,10a-tetrahydro-6H-benzo[c]chromene-1-ol.

75. The composition of any one of claims 45-52, wherein the active ingredient is 6,6,9-trimethyl-3-propyl-6a 7,10,10a-tetrahydro-6H-benzo[c]chromene-1-ol.

76. The composition of any one of claims 45-52, wherein the active ingredient is 6,6,9-trimethyl-3-propyl-6H-benzo[c]chromene-1-ol.

77. The composition of any one of claims 45-52, wherein the active ingredient is curcumin.

78. The composition of any one of claims 45-52, wherein the active ingredient is a flavonoid.

79. The composition of any one of claims 45-52, wherein the active ingredient is an anthocyanin or anthocyanidin.

80. The composition of any one of claims 45-52, wherein the active ingredient is epigallocatechin gallate.

81. The composition of any one of claims 45-52, wherein the active ingredient is a tannin.

82. The composition of any one of claims 45-52, wherein the active ingredient is a cannabinoid.

83. The composition of any one of claims 45-52, wherein the active ingredient is tetrahydrocannabinol.

84. The composition of any one of claims 45-52, wherein the active ingredient is tetrahydrocannabivarin.

85. The composition of any one of claims 45-52, wherein the active ingredient is delta8-tetrahydrocannabinol.

86. The composition of any one of claims 45-52, wherein the active ingredient is delta8-tetrahydrocannabivarin.

87. The composition of any one of claims 45-52, wherein the active ingredient is cannabidiol.

88. The composition of any one of claims 45-52, wherein the active ingredient is cannabidivarin.

89. The composition of any one of claims 45-52, wherein the active ingredient is cannabigerol.

90. The composition of any one of claims 45-52, wherein the active ingredient is cannabigerovarin.

91. The composition of any one of claims 45-52, wherein the active ingredient is cannabinol.

92. The composition of any one of claims 45-52, wherein the active ingredient is cannabivarin.

93. The composition of any one of claims 1-92, wherein the composition lacks triglycerides at a concentration greater than 0.1 percent by mass.

94. The composition of any one of claims 1-93, wherein the solid phase comprises carbonate; and the liquid phase comprises carbonate and bicarbonate.

95. The composition of any one of claims 1-94, wherein the composition is formulated for oral administration.

96. The composition of any one of claims 1-95, for use to manufacture a medicament.

97. A method to administer an active ingredient, comprising providing a composition according to any one of claims 1-96, wherein the composition comprises the active ingredient; and orally administering the composition.

98. The method of claim 97, comprising combining the composition with a second composition prior to administering the composition, wherein the second composition is a liquid that comprises water at a concentration of at least 50 molar.

99. A method to prepare a beverage, comprising providing a composition according to any one of claims 1-96; and combining the composition with a second composition, wherein the second composition is a liquid that comprises water at a concentration of at least 50 molar.

100. The method of claim 99, wherein the composition comprises an active agent; the active agent is dissolved in the liquid phase of the composition; and combining the composition with a second composition results in phase separation of the active agent from the water.

101. The method of any one of claims 98-100, comprising combining at least 100 milligrams and no greater than 5 grams of the composition with at least 10 grams and no greater than 800 grams of the second composition.

102. The method of any one of claims 98-101, wherein the second composition is a beverage.

103. The method of any one of claims 98-102, wherein a human performs the method; and the human consumes the combination of the composition and the second composition by drinking it.

104. A container that contains at least 100 milligrams and no greater than 5 grams of a composition according to any one of claims 1-96.

105. The container of claim 104, wherein the container is a sachet; and the composition is hermetically sealed within the container.

106. The container of claim 104 or 105, wherein the container comprises metallized polyethylene terephthalate that is metallized with aluminum.
